(19) United States
(12) Patent Application Publication
(10) Pub. No.: US 2026/0215958 A1
(43) Pub. Date: Jul. 30, 2026

(54) SLEEPING BOARD WITH ADJUSTABLE ELEVATION PROFILE FOR CHANGING SLEEPING POSITION TO REDUCE SNORING AND SLEEP APNEA

(71) Applicant: Dazhai HUANG, Irvine, CA (US)

(72) Inventor: Dazhai HUANG, Irvine, CA (US)

(21) Appl. No.: 19/459,668

(22) Filed: Jan. 26, 2026

Related U.S. Application Data
(60) Provisional application No. 63/774,582, filed on Mar. 19, 2025.
(60) Provisional application No. 63/750,170, filed on Jan. 27, 2025.

(51) Int. Cl. A61F 5/56

(52) U.S. Cl. CPC A61F 5/56

(57) ABSTRACT

A sleeping board to be placed on top of a bed, capable of changing the left-right elevation profile of the sleeping surface to help the user change their sleeping position to reduce snoring. The sleeping board includes at least two pivotable board sections arranged side-by-side in a transvers direction, which are independently pivotable relative to each other around one or more longitudinal pivotal axes, and two lifting and lowering mechanisms each attached to one of the at least two pivotable board sections to respectively change their pivotal angles. The lifting and lowering mechanisms may employ a torsion spring and pull string mechanism, a compression spring and pull string mechanism, a jack, a rotation and sliding arm, or a pair of rotation arms.

### BACKGROUND OF THE INVENTION

This invention relates to a sleeping board, and in particular, it relates to a sleeping board with adjustable elevation profile for changing a user’s sleeping positions to reduce snoring and sleep apnea.

Snoring and sleep apnea can lead to various health problems. The onset and severity of snoring are often related to the sleeping position; for example, people tend to snore less when sleeping on their sides as compared to on their backs. Beds with air mattresses designed to reduce snoring by changing the users’ sleeping position have been disclosed.

Beds used in hospitals and other healthcare settings, as well as some beds for home use, have sections that are pivotable relative to each other along pivotal axes that extend in the transverse direction of the bed, so that, for example, the upper (head) section of the bed can be tilted upwardly to create an inclined head section to help the user raise their upper body.

### SUMMARY OF THE INVENTION

The present invention is directed to a sleeping board that can help the user change their sleeping position to reduce sleep disruption and improve sleep quality. The sleeping board can be placed on top of a normal bed.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a sleeping board which includes: at least two pivotable board sections arranged side-by-side in a transvers direction of the sleeping board, wherein the two pivotable board sections are independently pivotable relative to each other around one or more pivotal axes that extend in the longitudinal direction of the sleeping board; and two lifting and lowering mechanisms each attached to one of the at least two pivotable board sections to respectively change their pivotal angles. The lifting and lowering mechanism may be implemented using torsion springs and a motor-driven pull string, or compression springs and a motor-driven pull string, or a motor-driven jack, or a motor-driven rotation and sliding arm with or without a biasing spring, or a pair of motor-driven rotation arms with or without a biasing spring, etc.

It is to be understood that both the foregoing general description and the following detailed description are exemplary and explanatory and are intended to provide further explanation of the invention as claimed.

### BRIEF DESCRIPTION OF DRAWINGS

FIGS. 1A-1C are cross-sectional views schematically illustrating portions of a sleeping board according to embodiments of the present invention.

FIGS. 2A-2B are top plan views schematically illustrating two pivotable board sections of a sleeping board according to embodiments of the present invention.

FIG. 3 schematically illustrates two pivotable board sections of a sleeping board according to an embodiment of the present invention.

FIGS. 4A-4B schematically illustrate the sleeping board in Figa. 1A-1C being operated to change a user’s sleeping position.

FIGS. 5 and 5A schematically illustrate portions of a sleeping board employing a torsion spring and pull string as the lifting and lowering mechanism according to an embodiment of the present invention.

FIGS. 6 and 6A schematically illustrate portions of a sleeping board employing a compression spring and pull string as the lifting and lowering mechanism according to another embodiment of the present invention.

FIGS. 7 and 7A schematically illustrate portions of a sleeping board employing a jack as the lifting and lowering mechanism according to another embodiment of the present invention.

FIGS. 8A-8B schematically illustrate portions of a sleeping board employing a rotating and sliding arm as the lifting and lowering mechanism according to another embodiment of the present invention.

FIGS. 9A-9D schematically illustrate portions of a sleeping board employing two pairs of rotating arms as the lifting and lowering mechanism according to another embodiment of the present invention.

FIG. 10 is a schematical block diagram illustrating a control system for sleeping board according to an embodiment of the present invention.

### DETAILED DESCRIPTION OF THE INVENTION

Embodiments of the present invention provide a sleeping board capable of changing the left-right elevation profile of the sleeping surface to help the user change their sleeping position to reduce snoring. The sleeping board may be used together with a normal bed, but it is a stand-alone unit that may be placed on top of a bed or other flat surface. The size of the board may be, for example, about 2 meters long and 1 meter wide. It may have a thickness of about 0.05-0.1 meter, or any other suitable thickness. The top layer of the sleeping board may include a soft or cushioning material to provide comfort, but the board sections are otherwise constructed of rigid materials.

The sleeping board is divided into two or more sections; at least two of the sections are disposed side-by-side in the transvers (left-right) direction of the sleeping board and are independently pivotable relative to each other (or to the rest of the board) around one or more pivotal axes that extend in the longitudinal direction of the sleeping board. The longitudinal direction is the direction along the larger dimension of the sleeping board, and the transvers direction is the direction along the smaller dimension of the sleeping board.

In one embodiment, schematically shown in FIG. 2A (top plan view), the entire sleeping board is divided into two pivotable board sections 11A and 11B, where each board section extends in the longitudinal direction of the sleeping board for the full length. The two board sections are arranged side-by-side across the transvers direction of the sleeping board and are independently pivotable around longitudinal axes (or a common axis) located near the left-right center of the sleeping board. In another embodiment, schematically shown in FIG. 2B (top plan view), the sleeping board is divided into an upper (head) board section 11C, a middle (torso) board section and a lower (leg) board section 11B, and the middle board section is further divided into two independently pivotable board sections 11A and 11B arranged side-by-side across the transvers direction. The upper and lower board sections 11C and 11D are stationary. In another embodiment (not shown in the drawings), the sleeping board is divided longitudinally into only an upper (head and torso) board section and a lower (leg) board section, and the upper (head and torso) board section is further divided into two independently pivotable board sections arranged side-by-side across the transvers direction.

A modified embodiment (not shown in the drawings) is similar to the embodiment of FIG. 2B but the middle (torso) board section is further divided into three board sections arranged side-by-side across the transvers direction, with the left board section and right board section independently pivotable and the center board section remaining stationary. Similar modifications may be made in the embodiment where the sleeping board is undivided in the longitudinal direction (FIG. 2A) or divided into two board sections in the longitudinal direction.

In another embodiment, which may also be represented by FIG. 2B, the upper (head) section 11C and the lower (leg) section 11D are not formed of rigid boards, but are formed of foam materials, or they may be a frame structure covered by foam materials, or other suitable structures and materials. Sections 11C and 11D are of appropriate thickness so that the entire sleeping surface is flat when the pivotable board sections 11A and 11B are level.

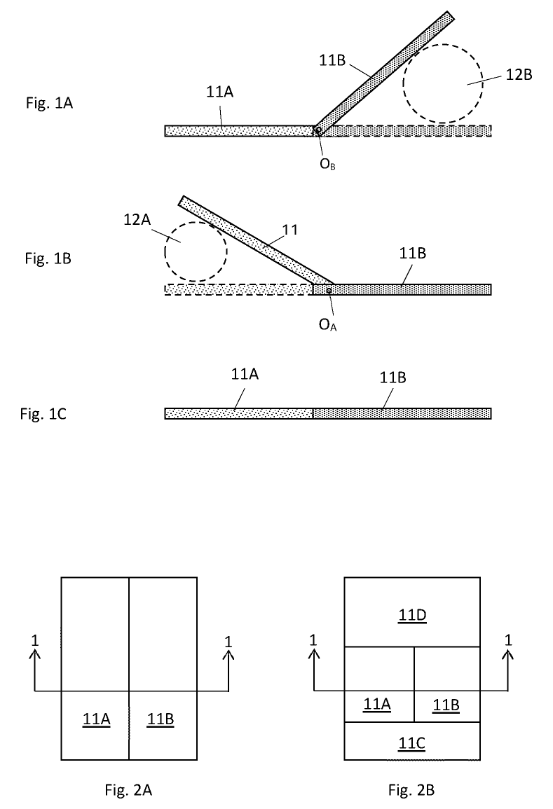

FIGS. 1A-1C schematically illustrate elevation profiles of the sleeping board with the two independently pivotable board sections 11A and 11B at different inclination angles, in cross-sectional views viewed along the horizontal longitudinal (head to foot) direction of the sleeping board (e.g., as indicated by arrows 1-1 in FIGS. 2A and 2B). In FIG. 1A, the right board section 11B is inclined upwards and the left board section 11A is horizontal; in FIG. 1B, the left board section 11A is inclined upwards and the right board section 11B is horizontal; and in FIG. 1C, neither board section is inclined and the sleeping board is flat and level. The pivotal axis O_{A} of the left board section 11A and the pivotal axis O_{B} of the right board section 11B may coincide in space or be separate in space (parallel to each other). Lifting and lowering mechanisms 12A and 12B are respectively provided for the corresponding board sections 11A and 11B to change their pivoting angles.

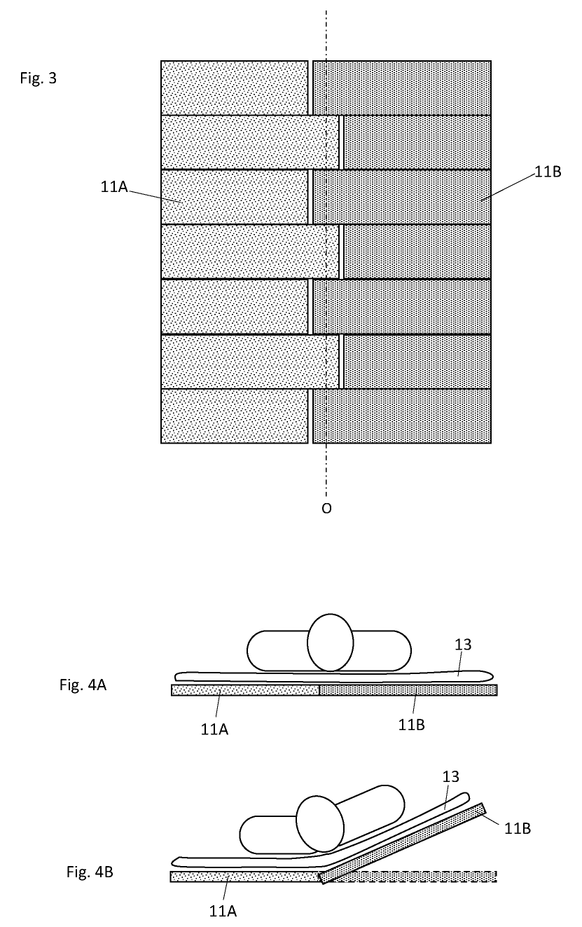

In one embodiment, shown in FIG. 3, the left and right board sections 11A and 11B have an interleaved or staggered structure where they meet each other, i.e., the two board sections have protrusions that alternate along the longitudinal direction, and the two board sections are pivotable around the same pivotal axis O.

FIG. 4A shows a user sleeping on the board that is flat, and FIG. 4B shows the right board section 11B being pivoted upwards and the user is gently pushed to turn to their left. A cushion 13 may be provided over the sleeping board to provide more comfort to the user, as shown in FIGS. 4A and 4B.

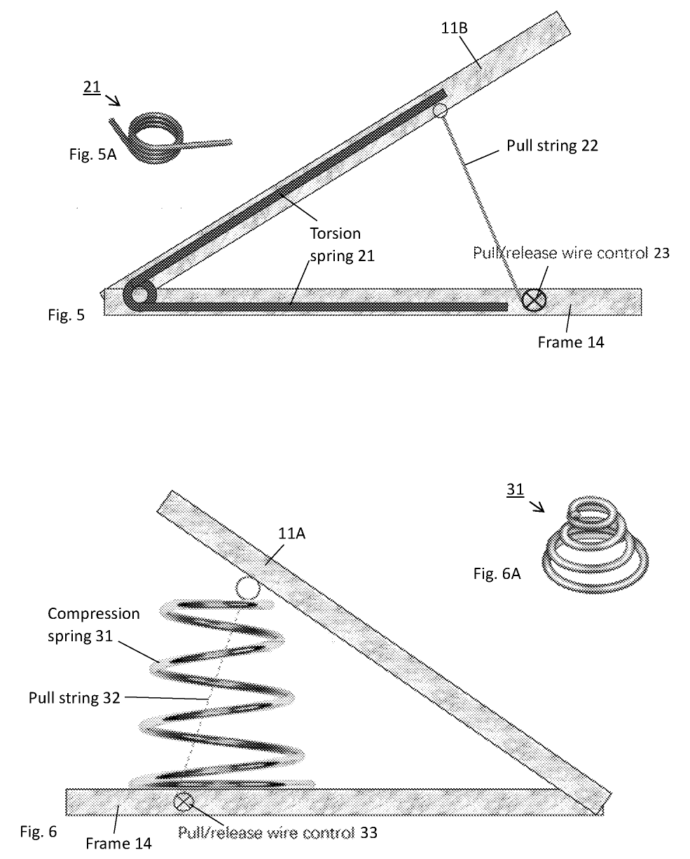

FIG. 5 (a view along the longitudinal direction) schematically illustrates an example of the lifting and lowering mechanism 12A (or 12B) which uses a torsion spring 21 and a pull string 22. The torsion spring 21 is attached to the pivotable board section 11B and a frame 14 of the sleeping board, located below the pivotable board section 11B, and is configured to urge the pivotable board section 11B away from the frame 14 into a tilted (i.e. inclined) position. In the illustrated embodiment, the center of the coil of the torsion spring approximately coincides with the pivoting axis of the pivotable board section 11B, but this is not required. In preferred embodiment, the torsion spring 21 is a coil with two arms (see FIG. 5A), and the two arms are embedded in the frame 14 and the pivotable board section 11B so that the pivotable board section 11B can be lowered completely into the same level as the frame 14. The frame 14 may be a portion of the stationary sections 11C and/or 11D in the embodiment of FIG. 2B, or a frame that surrounds the board sections 11A and 11B or surrounds the entire sleeping surface in the embodiments of FIG. 2A and FIG. 2B, or other suitable frame structure.

The pull string 22 is attached to the pivotable board section 11B and to the frame 14 via a spool 23. The spool 23 is controlled by a motor (not shown) to take in or release the string 22 to control the tilt angle of the pivotable board section 11B. Preferably, the distance between the pivotal axis and the attachment point of the string 22 on the board section 11B and the distance between the pivotal axis and the attachment point of the spool 23 on the frame 14 are approximately equal, but this is not required.

FIG. 6 (a view along the longitudinal direction) schematically illustrates another example of the lifting and lowering mechanism 12A (or 12B) which uses a compression spring 31 and a pull string 32. The compression spring 31 is attached to the pivotable board section 11A and a frame 14 of the sleeping board, and is configured to urge the pivotable board section 11A away from the frame 14 into a tilted (i.e. inclined) position. In preferred embodiments, the compression spring is a conical spiral coil (see FIGS. 6 and 6A) that can be compressed into a flat spiral coil with a low profile, and the compressed coil is partially or fully disposed within a recess of the pivotable board section 11A and/or the frame 14. The frame 14 may be a portion of the stationary sections 11C and/or 11D in the embodiment of FIG. 2B, or a frame that surrounds the board sections 11A and 11B or surrounds the entire sleeping surface in the embodiments of FIG. 2A and FIG. 2B, or other suitable frame structure. The frame 14 may also include a flat bottom board disposed below the pivotable board sections 11A and 11B, and may have a recess to partially or fully accommodate the compressed coil. Preferably, in the fully compressed state, the compression spring 31 is entirely disposed in the recesses of the pivotable board section 11A and/or of the frame so that the sleeping board can be level and flat as shown in FIG. 1C. Multiple springs 31 may be provided at different locations (e.g. one or more rows of springs, multiple springs arranged in a 3x3, 2x2, 2x3 or 3x2 grid pattern, etc.) to improve user experience and comfort.

The pull string 32 is attached to the pivotable board section 11A and to the frame 14 via a spool 33. The spool 33 is controlled by a motor (not shown) to take in or release the string 32 to control the tilt angle of the pivotable board section 11A. Preferably, the distance between the pivotal axis and the attachment point of the string 32 on the board section 11A and the distance between the pivotal axis and the attachment point of the spool 33 on the frame 14 are approximately equal, but this is not required.

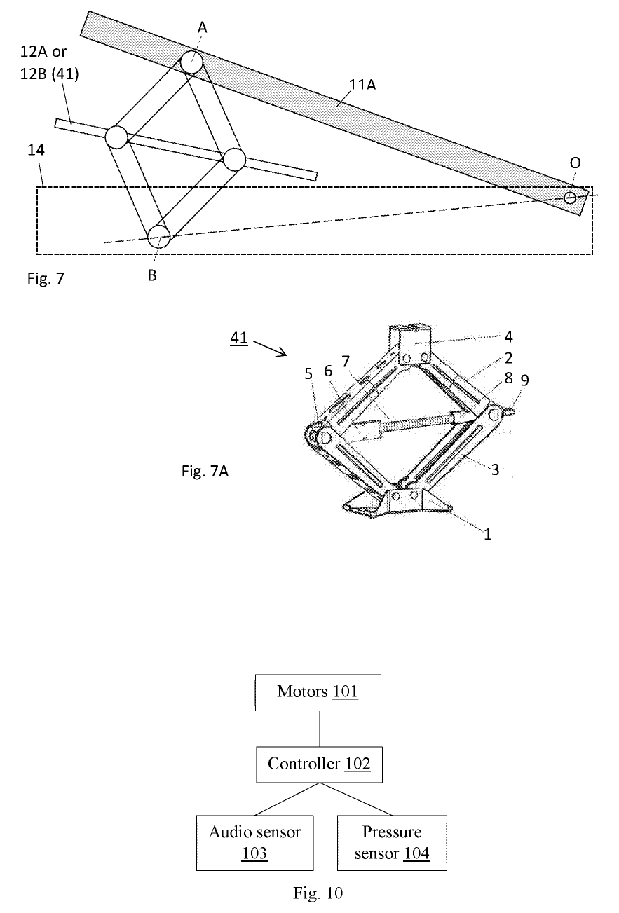

FIG. 7 (a view along the longitudinal direction) schematically illustrates yet another example of the lifting and lowering mechanism 12A (or 12B) which uses a jack 41 to raise and lower the end of the pivotable board section 11A (or 11B). The jack 41 may have a structure similar to a conventional jack shown in FIG. 7A, and is attached to the pivotable board section 11A at a point A, and to a frame 14 of the sleeping board at a point B, where the distances of points A and B to the pivotal axis O of the pivotable board section are approximately equal. The frame 14 is designed to accommodate the jack 41 when the jack is in the retracted (lowest profile) state, so that the pivotable board section 11A can be level. Because of the size of the jack in the retracted state, the attachment point B may be lower than the pivotal axis O to allow attachment point A to be level with the pivotal axis O when the jack is in the retracted state.

In the example shown in FIG. 7A, the jack includes four arms 2, 3 forming a diamond shape (a rhombus). The upper and lower ends 4 and 1 of the diamond shape are respectively attached to the pivotable board section 11A and the frame 14 at points A and B as shown in FIG. 5. The two middle connecting points 5 of the diamond shape are connected to each other by a drive shaft 6, 7, 8, 9, formed of a threaded shaft 7 and a threaded nut 8. Turning the nut 8 (e.g. by a handle or know 9 at its end) will change the distance between the two middle connecting points 5, thereby changing the height between the upper end 4 and lower end 1. The jack is driven by a suitable motor (not shown in the drawings).

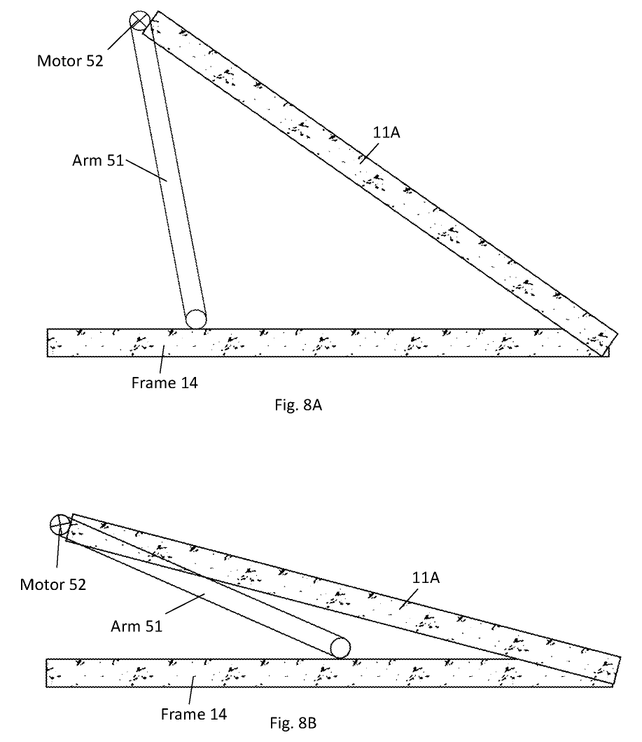

FIGS. 8A and 8B (views along the longitudinal direction) schematically illustrate yet another example of the lifting and lowering mechanism 12A (or 12B) which uses a rotating and sliding arm 51. The arm 51 is located between the pivotable board section 11A and a frame 14 of the sleeping board; the upper end of the arm is pivotally mounted on the pivotable board section 11A via a motor 52, preferably at a location near the outer edge of the board section, while the lower end of the arm is slidable along the frame, for example, via a roller wheel on the arm and a sliding rail or sliding slot on the frame (not shown). The motor 52 drives the arm 51 to rotate, such that the lower end of the arm travels freely to different positions along the frame 14, as shown in FIGS. 8A and 8B. This results in different tilt angles of the board section 11A.

In an alternative embodiment (not shown), a linear actuator is employed to drive the lower end of the arm 51 along the frame 14, while the upper end of the arm is pivotally mounted on the board section 11A to freely rotate without a motor. The linear movement of the lower end of the arm results in different tilt angles of the board section 11A. Further, it is possible to use two or more sets of arms and corresponding motors, spaced apart in the longitudinal direction.

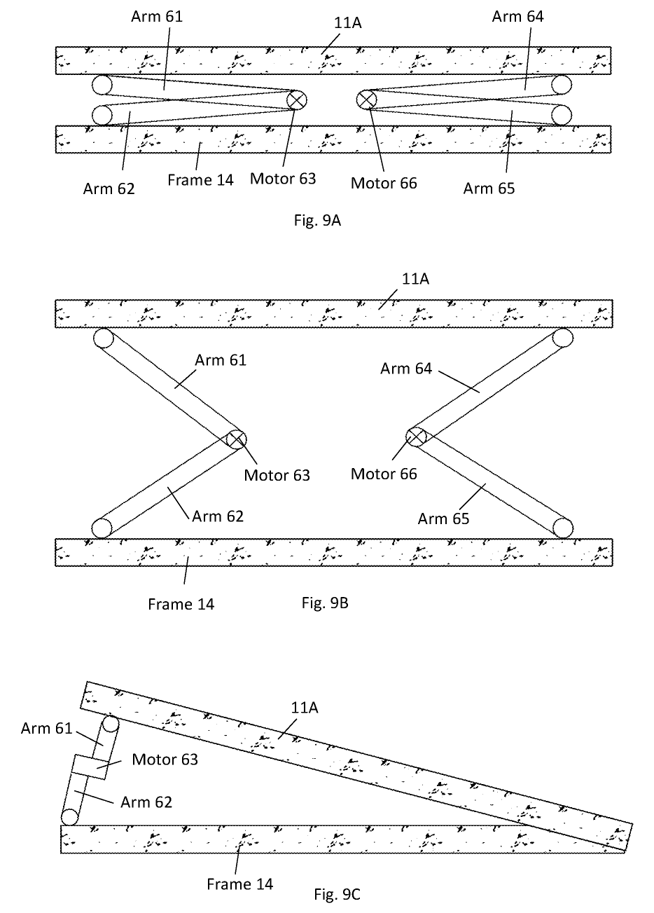

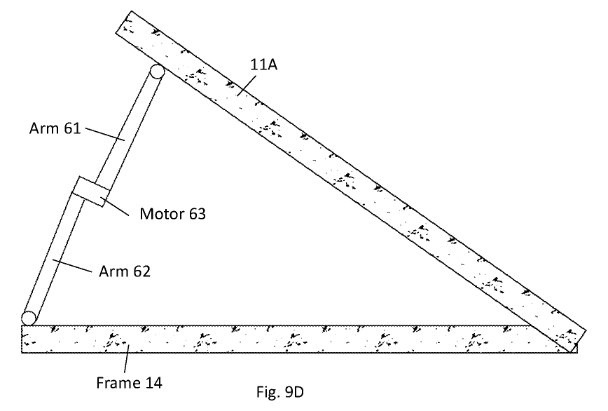

FIGS. 9A-9D schematically illustrate yet another example of the lifting and lowering mechanism 12A (or 12B) which uses one pair or two pairs of connected rotating arms. FIGS. 9A and 9B are views from the side, where the outer longitudinal edges of the board section 11A and frame 14 are seen. FIGS. 9C and 9D are views along the longitudinal direction. The pair of arms 61 and 62 are located between the pivotable board section 11A and the frame 14 of the sleeping board. The upper end of the first arm 61 and the lower end of the second arm 62 are pivotally mounted on the board section 11A and the frame 14, respectively, near their outer edges. The other ends of the arms 61 and 62 are joined together via a motor 63, which drives the two arms to rotate relative to each other. As the motor drives the arms to rotate relative to each other, the outer edge of the board section 11A is lifted or lowered, thereby changing the tilt angle of the board section 11A. FIGS. 9A and 9C show the pivotable board section 11A at a relatively small tilt angle, and FIGS. 9B and 9D show it at a relatively large tilt angle.

The arms 64 and 65 and motor 66 are similarly structured as the arms 61 and 62 and the motor 63, where the two sets of arms and motor structures 61-63 and 64-66 are spaced apart in the longitudinal direction. In an alternative embodiment, only one set of arms and one motor is provided.

One or two biasing springs may be additionally provided between and affixed to the board section 11A and the frame 14, which cooperate with the motors 63 and 66 and provide a part of the required force to tilt the board section 11A. Each biasing spring may be a compression spring, similar to the spring 31 shown in FIGS. 6 and 6A. Alternatively, each biasing spring may be a torsion spring similar to the spring 21 shown in FIG. 5A, but preferably (unlike in FIG. 5), the two arms of the torsion spring are disposed next to and approximately parallel to the arms 61 and 62 (or 64 and 65), with the coil portion of the torsion spring disposed approximately on the same axis as the rotation axis where the arms 61 and 62 (or 64 and 65) are connected to each other. The torsion spring may alternatively be arranged in other orientations.

The parameters of the spring are preferably set such that: when the board section 11A is level (i.e. at zero degree tilt angle), the biasing spring is compressed and exerts an upward force that is equal to approximately one half of the total upward force required to push the board section 11A to tilt upwards from the level position when an average-sized user is sleeping on the sleeping board; and at the designed maximum tilt angle, the biasing spring is compressed to a lesser extent, or substantially uncompressed, or may even be slightly stretched. During lifting (with the user sleeping on the board), the motor provides the remaining amount of the required total upward force. Note that because only part of the user’s body is located over the board section 11A, the required total upward force at zero degree tilt angle is only a fraction of the weight of the user. The required total upward force tends to be the largest at zero degree tilt angle, and becomes smaller at larger tilt angles, partly because the user may be turning away from the board section 11A. Thus, the cooperation of the biasing spring and the motor is advantageous in that the spring provides larger amounts of upward force at lower tilt angles when larger amounts of total force are required. At zero degree tile angle, the motor needs to provide approximately one half of the required total upward force. When the board section 11A is to be lowered, assuming the user is not sleeping over that board section, the motor will apply a downward force to compress the biasing spring; the largest amount of the downward force required will be when the board section 11A is back to zero degree tilt angle. Of course, the actual amount of force required from the motor during actual operations will vary depending on many factors, but the biasing spring may help to reduce the maximum amount of force required from the motor by approximately one half. This allows for a smaller motor to be used.

A biasing spring may similarly be provided in the embodiment shown in FIGS. 8A-8B.

In all of the above embodiments, the term “motor” should be understood to refer to a motor assembly that includes a motor (preferably a stepper motor) and a suitable gear assembly that transmits the rotation of the motor to the rotation of corresponding parts (the spools 23 and 33, the jack 41, the arm 51, and the arms 61, 62 and 64, 65). The gear assembly is designed such that the total rotation range of the corresponding parts corresponds to multiple (tens, hundreds, or even more) revolutions of the motor. This reduces the required power of the motor, and is suitable for the intended application of the sleeping board system, which is to slowly tilt the pivotal board sections, e.g., to move from zero tile to the maximum tilt angle over a time period of a half to several minutes. Such a motor assembly has the advantages of low noise and smooth operation. It also reduces the size and power requirement of the motor and thereby lowering cost.

In all of the above embodiments, the frame may be formed of metal bars, optionally covered with soft padding.

In all of the above embodiments, the cushion or mattress 13 may be provided with heating and/or cooling elements to improve comfort of the user.

FIG. 10 is a schematic block diagram showing various components of the sleeping board and its control system. The components are communicatively coupled to each other by wired or wireless communication channels. In preferred embodiments, the lifting and lowering mechanisms 12A and 12B are operated by electric motors 101 which are controlled by a controller 102. The motors may be powered by battery or plug-in electricity. Preferably, the operation of the lifting and lowering mechanism to change the profile of the sleeping board is performed only if snoring is detected. To this end, an audio sensor 103 is provided adjacent to the bed to sense the snoring sound of the user. When the user is not snoring, the lifting and lowering mechanism are maintained in the flat configuration as shown in FIG. 1C. When snoring is detected by the audio sensor 103, one of the lifting and lowering mechanisms 12A and 12B is operated to tilt the pivotable board sections 11A or 11B upwards, thereby creating the sloped profile to help the user change their sleeping position.

Optionally, pressure sensors 104 may be provided at multiple points over the sleeping board to detect the user’s sleeping position (e.g., on their back or on their side, and which side). The information sensed by the pressure sensors 104 may be used by the controller 102 to control the operation of the lifting and lowering mechanisms 12A and 12B (once the sound sensor detects snoring), e.g., to determine whether the left lifting and lowering mechanism 12A or the right lifting and lowering mechanism 12B should be operated and how much the pivotable board sections 11A or 11B should be tilted, etc.

The controller 102 may also be coupled to a user interface device (not shown), such as a control panel (e.g., a panel mounted on the side of the bed or on the wall of the bedroom), a handheld device (e.g., a remote control device, a smart phone, etc.), etc., which allows the user to interact with the controller. The controller 102 may include a microcontroller or any other suitable electronic circuits.

While the sleeping board described above is intended to be a stand-alone structure to be placed on top of a bed, it may also be integrated into the bed itself. For example, the frames may be a part of the bed, or the pivotal axes may be fixed to the bed, or the stationary sections of the sleeping board may be a part of the bed, etc.

It will be apparent to those skilled in the art that various modification and variations can be made in the sleeping board of the present invention without departing from the spirit or scope of the invention. Thus, it is intended that the present invention cover modifications and variations that come within the scope of the appended claims and their equivalents.

1. A sleeping board, comprising:
at least two pivotable board sections arranged side-by-side in a transvers direction of the sleeping board, wherein the two pivotable board sections are independently pivotable relative to each other around one or more pivotal axes that extend in the longitudinal direction of the sleeping board; and
two lifting and lowering mechanisms each attached to one of the at least two pivotable board sections to respectively change their pivotal angles.

2. The sleeping board of claim 1, further comprising one or more stationary sections disposed adjacent to the two pivotable board sections in a longitudinal direction.

3. The sleeping board of claim 1, wherein each of the two lifting and lowering mechanisms includes:
one or more torsion springs, located below the corresponding pivotable board section and attached to the pivotable board section and a frame of the sleeping board, and configured to urge the pivotable board section away from the frame into a tilted position;
a pull string, attached to the pivotable board section and to the frame via a spool; and
a motor configured to control the spool to take in or release the string to change a tilt angle of the pivotable board section.

4. The sleeping board of claim 1, wherein each of the two lifting and lowering mechanisms includes:
one or more compression springs, located below the corresponding pivotable board section and attached to the pivotable board section and a frame of the sleeping board, and configured to urge the pivotable board section away from the frame into a tilted position;
a pull string, attached to the pivotable board section and to the frame via a spool; and
a motor configured to control the spool to take in or release the string to change a tilt angle of the pivotable board section.

5. The sleeping board of claim 4, wherein each of the two lifting and lowering mechanisms includes multiple compression springs arranged in a row or a grid pattern.

6. The sleeping board of claim 1, wherein each of the two lifting and lowering mechanisms includes a jack, which is located below the corresponding pivotable board section and attached to the pivotable board section and a frame of the sleeping board, and is configured to change a tilt angle of the pivotable board section.

7. The sleeping board of claim 1, wherein each of the two lifting and lowering mechanisms includes:
a rotation and sliding arm, located below the corresponding pivotable board section, having a first end pivotally connected to the pivotable board section and a second end slidably connected to a frame of the sleeping board; and
a motor connected to the first end of the rotation and sliding arm, configured to drive the rotation and sliding arm to rotate relative to the pivotable board section.

8. The sleeping board of claim 7, wherein each of the two lifting and lowering mechanisms further includes a biasing spring, located below the corresponding pivotable board section and attached to the pivotable board section and the frame of the sleeping board, and configured to urge the pivotable board section away from the frame into a tilted position.

9. The sleeping board of claim 1, wherein each of the two lifting and lowering mechanisms includes:
a first rotation arm and a second rotation arm, located below the corresponding pivotable board section, wherein a first end of the first rotation arm is pivotally mounted on the pivotable board section, a first end of the second rotation arm is pivotally mounted on a frame of the sleeping board, and a second end of the first rotation arm is pivotally connected to a second end of the second rotation arm; and
a motor connected to the second end of the first rotation arm and the second end of the second rotation arm, configured to drive the first and second rotation arms to rotate relative to each other.

10. The sleeping board of claim 9, wherein each of the two lifting and lowering mechanisms further includes a biasing spring, located below the corresponding pivotable board section and attached to the pivotable board section and the frame of the sleeping board, and configured to urge the pivotable board section away from the frame into a tilted position.
